(19) United States
(12) Patent Application Publication
(10) Pub. No.: US 2008/0207777 A1
(43) Pub. Date: Aug. 28, 2008

(54) Emulsifier System, Emulsion and the Use Thereof

(75) Inventors: Helmut Auweter, Limburgerhof (DE); Heribert Bohn, Wattenheim (DE); Christian Kopsel, Weinheim (DE)

Correspondence Address: CONNOLLY BOVE LODGE & HUTZ, LLP, P O BOX 2207, WILMINGTON, DE, 19899, US

(73) Assignee: BASF Altiengesellschaft Patents, and Trademarks and Licenses, Ludwigshafen (DE)

(21) Appl. No.: 11/994,515

(22) PCT Filed: Jun. 29, 2006
(86) PCT No.: PCT/EP2006/063665

§ 371 Date: Mar. 6, 2008

(30) Foreign Application Priority Data
Jul. 4, 2005 (DE) 10 2005 031 464.3

(51) Int. Cl. A61K 9/107; A23L 1/00; A23L 1/48; B01F 17/00; A61K 8/06; A61K 47/14; A61K 8/37

(52) U.S. Cl. 514/785; 426/564; 516/73

(57) ABSTRACT

The present invention relates to an emulsifier system comprising a) an ascorbic ester with long-chain fatty acids, b) an ethoxylated sorbitan fatty acid ester and c) a sugar fatty acid ester and emulsions prepared therewith.

The present invention relates to an emulsifier system which comprises a) an ascorbic ester with long-chain fatty acids, b) an ethoxylated sorbitan fatty acid ester and c) a sugar fatty acid ester. The total of components a), b) and c) is 100% by weight.

The present invention further relates to an emulsion which comprises α) a disperse phase comprising a fat-soluble substance α₁), β) glycerol or glycerol mixed with water as dispersion medium and γ) the inventive emulsifier system. The total of components α), β), γ) is 100% by weight based on the emulsion.

The present invention further relates to a process for preparing the inventive emulsion, and the use thereof in human nutrition, in animal nutrition, in cosmetics or in the pharmaceutical industry. Further embodiments of the present invention are to be inferred from the claims, the description and the examples. It will be appreciated that the features to which the invention relates and which are mentioned above and will be explained hereinafter can be used not only in the combination indicated in each case, but also in other combinations, without departing from the scope of the invention.

German patent DE 2 363 534 describes the preparation of sugar-containing liquid vitamin and carotenoid products. Physiologically acceptable emulsifiers such as lecithin or ascorbyl palmitate are used for the emulsification. One disadvantage of such products is the tendency of the sugar or sugar alcohol to crystallize, e.g. on storage of the product at low temperatures. This tendency to crystallize leads to unwanted inhomogeneites.

European patent EP 551 638 relates to the stabilization of liquid products of fat-soluble substances over a maximum storage period (>6 months). The esters of ascorbic acid with long-chain fatty acids are proposed as emulsifier for this purpose. However, the emulsions require cool storage. This may increase the transport costs and make handling difficult.

Japanese patent JP 2 000 212 066 discloses the use of polyoxyethylene sorbitan fatty acid esters and of sugar fatty acid esters as emulisifier for emulsions of fat-soluble substances.

Japanese patent JP 08 120 187 discloses the use of sorbitan fatty acid esters and sucrose fatty acid esters as emulsifier for carotenoid emulsions.

Korean patent KR 20 020 018 518 discloses the use of sucrose fatty acid esters and of sorbitan fatty acid esters as emulsifier for a carotenoid emulsion. Concentrations of up to 3% β-carotene are achieved.

European patent EP 1 095 986 deals with the problem of preventing the coagulation of the disperse phase. This problem is solved by providing a method for stabilizing liquid, aqueous products of fat-soluble substances. This entails a protective colloid-free oil-in-water emulsion being mixed with a fat-soluble substance with a protective colloid. This preparation is blended with an aqueous phase. The protective colloid is a high molecular weight stabilizer. The problem of thermal and storage stability is not solved thereby.

European patent EP 361 928 shows an extremely finely divided emulsion with a glycerol/water mixture as dispersion medium and a fat-soluble medicament as disperse phase. Nonionic stabilizers having a molecular weight of 1000 or more are used as stabilizers. The average particle size of the disperse phase in this case is between 10 to 70 nm. The emulsions mentioned in the examples show changes after only 3 months. The problem of the thermal and storage stability is not solved thereby, because cooling may be necessary in this case too.

One of the objects of the present invention was to provide an emulsifier system which was intended to be employable as diversely as possible. It was particularly intended that the emulsifier system be usable for preparing emulsions which are physiologically tolerated. The emulsifier system was intended in particular to result in emulsions having the desired positive properties of
high concentration of the fat-soluble substance,
storage stability,
temperature insensitivity and
high stability and color intensity.

It was the particular object of the present invention to provide an emulsion having a high content of a fat-soluble substance, preferably amounting to 2.0% by weight or more, but if possible 10% by weight or more, of the emulsion. The emulsion was intended to have high storage stability so that storage is possible both at room temperature and elevated temperatures up to about 40° C. A further object of the present invention was to improve the stability of an emulsion on introduction into beverages, and to improve the storage stability in these beverages. The emulsion was in particular to be stable after introduction into beverages and after the pasteurization step. It was further desired that the emulsion of the invention has a high relative color intensity and color stability as required for example in the beverage industry. The emulsion was intended especially to be bacteriostatic for applications in the human or animal food, cosmetics or pharmaceutical industry.

This object has been achieved according to the invention by the emulsifier system described at the outset and by the emulsion described at the outset

Emulsifiers are aids for preparing and for stabilizing emulsions. By emulsions are meant liquid-liquid mixtures. By stabilization is meant the prevention of a disperse phase α) and a dispersion medium β) segregating to result in the thermodynamically stable final state. Disperse phase stands in connection with the present invention for the finely distributed phase of the emulsion. The continuous phase of the emulsion is designated the dispersion medium in connection with the present invention. The emulsifier system of the invention is employed as dispersant.

The emulisifier system of the invention comprises three emulsifiers. The emulsifier system of the invention may comprise further ingredients. All the ingredients of the emulsifier system of the invention together amount to 100% by weight based on the emulsifier system.

Ascorbic esters are employed as emulsifier a). Long-chain alkyl esters such as C₁₀- to C₂₀-alkyl esters are used according to the invention. A preferred embodiment of an emulsifier a) is the ascorbic ester with C₁₆- to C₁₈-alkyl radicals. In a particularly preferred embodiment, ascorbyl palmitate (C₁₆-alkyl ester) is employed as emulsifier a).

It is possible for one or more different emulsifiers a) to be present in the emulsifier system of the invention, for example two or three different emulsifiers a). This does not entail departing from the inventive concentration of an ester of ascorbic acid in the emulsifier system. Preferably only one emulsifier a), especially in the embodiment with ascorbyl palmitate, is employed for the emulsifier system of the invention.

The proportion of an emulsifier a) in the emulsifier system of the invention may vary within wide limits. An emulsifier a) is preferably present in a concentration of from 30% by weight to 70% by weight, particularly preferably in a concentration of from 40% by weight to 60% by weight, where the % by weight data are in each case based on the emulsifier system of the invention.

The effect of an emulsifier a), especially of ascorbyl palmitate as an emulsifier a), can be increased by forming a salt, usually an alkali metal salt, especially a sodium salt. For this purpose, sodium hydroxide solution is added, usually in the 0.5 to 2 molar quantity, preferably in the 1 molar quantity, to the ascorbic ester. It is particularly preferred to employ a mixture of ester and salt.

Generally suitable as an emulsifier b) are polyethylene glycol sorbitan fatty acid esters. An emulsifier b) generally has an HLB in the range from 10 to 18. Preferred emulsifiers b) have the general chemical structural formula

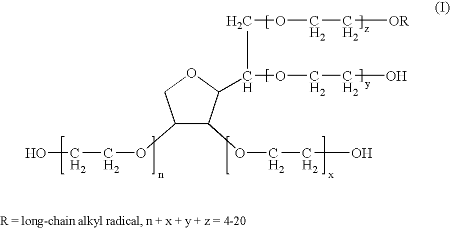

A long-chain alkyl radical generally means a C₁₀ to C₂₀-alkyl radical, and especially a C₁₆-C₁₈-alkyl radical

A preferred embodiment is an ethoxylated sorbitan oleic acid monoester. An emulsifier b) has, in a particularly preferred embodiment, the general chemical structural formula

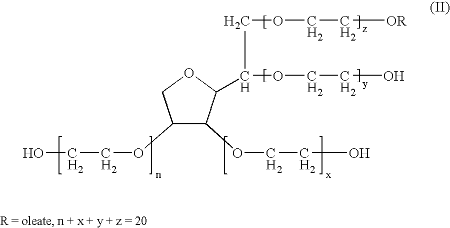

The proportion of monoester is generally 55% by weight or more, preferably from 70% by weight to 85% by weight and particularly preferably from 75% by weight to 85% by weight. An emulsifier b) is preferably nonionic.

It is possible for one or more different emulsifiers b) to be present in the emulsifier system of the invention, for example two or three different emulsifiers b). Preferably only one emulsifier b) is employed for the emulsifier system of the invention.

The proportion of an emulsifier b) in the emulsifier system of the invention can vary within wide limits. An emulsifier b) is preferably present in the concentration of from 10% by weight to 50% by weight, particularly preferably in a concentration of from 12.5% by weight to 40% by weight and especially in a concentration of from 20% by weight to 30% by weight, where the % by weight data are in each case based on the emulsifier system of the invention.

Generally suitable as an emulsifier c) are sugar fatty acid esters. An emulsifier c) generally has an HLB in the range from 10 to 18. Suitable examples are sucrose stearate, sucrose palmitate, sucrose myristate, sucrose laurate and sucrose oleate. In each of these cases the proportion of monoester is 55% by weight or more, preferably in the range from 70% by weight to 85% by weight, particularly preferably from 75% by weight to 85% by weight. Preferred emulsifiers c) have the general chemical structural formula

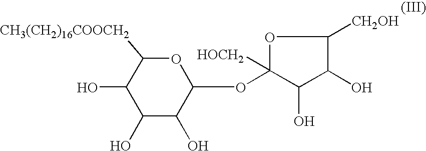

It is possible for one or more different emulsifiers c) to be present in the emulsifier system of the invention, e.g. two or three different emulsifiers c). Preferably only one emulsifier c) is employed for the emulsifier system of the invention.

The proportion of an emulsifier c) in the emulsifier system of the invention can vary within wide limits. An emulsifier c) is preferably present in a concentration of from 10% by weight to 50% by weight, particularly preferably in a concentration of from 12.5% by weight to 40% by weight and especially in a concentration of from 20% by weight to 30% by weight, where the % by weight data are in each case based on the emulsifier system of the invention.

One, two or all of the three emulsifiers a), b) and c) have in a particular embodiment a low molecular weight, for example a molecular weight below 2000 and in particular a molecular weight below 1000. In a particularly preferred embodiment, emulsifier a) has a low molecular weight. The emulsifier system of the invention facilitates in particular the distribution of the disperse phase α). Low molecular weight emulsifiers, in particular an emulsifier a), are distinguished by their rapid attachment, compared with the high molecular weight stabilizers, to the emulsion which has been generated for example by mechanical action. The low molecular weight emulsifiers can, however, also serve to stabilize the emulsion after preparation.

The emulsifier system of the invention is distinguished in particular by its synergistic effect. By synergy is meant the cooperation of different emulsifiers to a synchronized overall performance and in the context of the present invention also the overall performance, resulting therefrom, of the emulsifier system of the invention. This overall performance is greater than the total of the individual performances.

The emulsifier system of the invention is composed in a preferred embodiment of 20% by weight to 30% by weight of an emulsifier b) in the embodiment of an ethoxylated sorbitan fatty acid ester of the structural formula

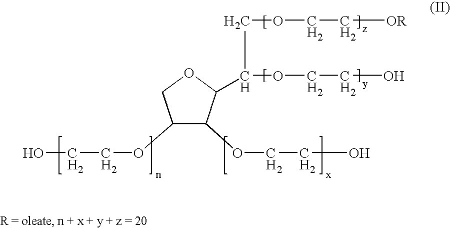

and 20% by weight to 30% by weight of an emulsifier c) in the embodiment of a sugar fatty acid ester having the chemical structural formula

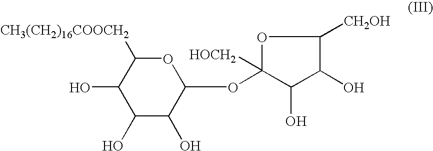

and an amount of ascorbyl palmitate such that the total reaches 100% by weight based on the emulsifier system.

Targeted selection and combination of the three emulsifiers a), b) and c) of the emulsifier system of the invention surprisingly leads to an emulsifier system with which it is possible for example to prepare a surprisingly advantageous emulsion of the invention as described below. The emulsifier system of the invention facilitates in this connection the preparation of the emulsion, the storage of the emulsion and the use of the emulsion.

An emulsion of the invention comprises a disperse phase α), a dispersion medium β) and γ) an emulsifier system of the invention. However, further components may also be present. All the ingredients of an emulsion of the invention together amount to 100% by weight based on the emulsion.

A fat-soluble substance α) present in the emulsion of the invention can in principle be any fat-soluble substance. A fat-soluble substance α₁) is preferably physiologically tolerated, i.e. it is tolerated in terms of human and in terms of animal physiology. However, a plant physiological tolerability is also possible. Suitable examples are the fat-soluble vitamins A, D, E or K or derivatives thereof, for example vitamin A esters and vitamin E esters such as retinyl acetate or tocopherol acetate, tocotrienol, vitamin K₁, vitamin K₂, xanthophylls, and carotenoids such as canthaxanthin, astaxanthin, zeaxanthin, lutein, lycopene, apocarotenal and especially β-carotene. Carotenoids are colored pigments which are widespread in nature and are present in extremely finely dispersed form in many foodstuffs and confer on the foodstuffs a characteristic color. Besides the generally known provitamin A effect of many carotenoids, for this reason carotenoids are also of interest as colorants for the human and animal food and pharmaceutical industries. The colors achieved by relatively concentrated carotenoid emulsions usually cover the range from orange to red. Relatively concentrated β-carotene-containing emulsions according to the present invention are by contrast distinguished by a brilliant yellow hue as is desired for many food products, and a high color strength.

Further fat-soluble substances α₁) which can be employed within the context of the present invention are polyunsaturated fatty acids such as, for example, arachidonic acid, docosahexaenoic acid, eicosapentaenoic acid, linoleic acid, linolenic acid, both in free form and as triglyceride, and aromatic oils such as orange oil, peppermint oil or citrus oils, for example one having the chemical structural formula

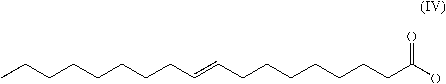

Further suitable fat-soluble substances α₁) are glycerides of polyunsaturated fatty acids, such as wheat oil, sunflower oil or corn oil or mixtures of said oils.

Finally, any very general fat-soluble substances α₁) which play a physiological role in the human or animal body and are, owing to their insolubility in water, usually processed to emulsions are suitable as ingredient of the emulsion.

Preferred fat-soluble substances α₁) in the context of the present invention are especially the abovementioned fat-soluble vitamins, e.g. vitamin A, D, E and K and derivatives thereof xanthophylls, and the polyunsaturated fatty acids and β-carotene. Particular preference is given to β-carotene.

A β-carotene as a fat-soluble substance α₁) is generally present dissolved in an oil. The β-carotene dissolved in an oil is generally employed together with the oil as disperse phase. Examples of suitable oils are all physiologically tolerated oils, especially peanut oil, sunflower oil, olive oil or other triacyl glycerides. The β-carotene is preferably dissolved in a triacyl glyceride having the general chemical structural formula

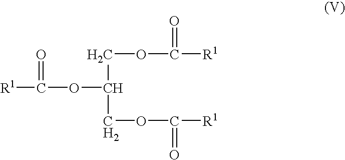

A β-carotene is in this case generally dissolved in suitable amounts of oil. This is generally the case with an oil to β-carotene ratio of from 1 to 3, preferably of from 1.5 to 2.5 and particularly preferably of from 1.8 to 2.2.

By a fat-soluble substance α₁) is initially meant a fat-soluble substance. However, it is also possible to combine various fat-soluble substances, for example two or three fat-soluble substances α₁). One fat-soluble substance α₁) is preferably employed.

A fat-soluble substance α₁) of the invention is preferably present in amounts of 40% by weight or less based on the emulsion. In a preferred embodiment, the proportion of a fat-soluble substance α₁) is from 2 to 40% by weight based on the emulsion. A fat-soluble substance α₁) can, however, also be present in concentrations of from 2% by weight to 20% by weight or of from 5% by weight to 15% by weight, particularly preferably, for example for applications in the beverage industry, 10% by weight, where the % by weight data are based in each case on the emulsion.

The average particle size of the disperse phase α) of the emulsion of the invention is generally 500 nm or less. In a further embodiment of the invention, the average particle size of the disperse phase α) is from 50 nm to 250 nm. In a preferred embodiment of the invention, the particle size of the disperse phase α) is from 50 nm to 100 nm. The average particle size of an emulsion of the invention can in this connection be measured for example via a mass-based weight distribution with a photon correlation spectrometer at a wavelength of 632.8 nm.

The dispersion medium β) generally accounts for from 40% by weight to 95% by weight, preferably 50% by weight to 80% by weight, of the emulsion. The dispersion medium β) may consist only of glycerol. However, 50% of water or less may also be 35 present in addition to the glycerol. The glycerol-water ratio is preferably from 95:5 to 85:15. The weight ratio of glycerol and water depends on the properties of the fat-soluble substance α₁) to be dispersed, and on the requirement for a uniform and fine distribution.

It is possible to employ distilled or otherwise deionized or partially deionized water. It is, however, also possible to employ drinking water.

The emulsifier system γ) accounts for in particular from 0.2 to 10% by weight of the emulsion. In another embodiment, the emulsifier system γ) of the invention accounts for from 0.3% by weight to 5% by weight based on the emulsion of the invention. In a preferred embodiment, the emulsifier system γ) of the invention accounts for from 0.5% by weight to 1.0% by weight based on the emulsion of the invention.

The emulsion of the invention shows in a particular embodiment of the present invention a high stability over a long storage period. An emulsion of the invention can be for example stored at room temperature and in particular at an elevated temperature of about 40° C. with substantial exclusion of light for approximately up to 2 years. By room temperature is meant in connection with the present invention temperatures from 15° to 35° C., but especially from 20° C. to 30° C. An emulsion of the invention generally remains stable over this storage period of up to 2 years. Coalescence of the disperse phase α) of an emulsion of the invention, especially ring formation, flocculation or color loss, do not generally occur.

The emulsion of the invention further shows a high color intensity. The measure used for the color intensity is the relative color intensity, also referred to as the E1/1 value. The E1/1 value defines the specific extinction of a 1.0% strength aqueous emulsion in a 1 cm cuvette at the absorption maximum. By a high color intensity is meant in connection with the present invention in particular a relative color intensity of 180 or more. The relative color intensity of the emulsion of the invention is usually from 180 to 200.

The emulsion of the invention exhibits in a further embodiment of the present invention an advantageous low turbidity, which is desired for example for certain food products. The turbidity of an emulsion is generally determined as specified in ISO 7027/DIN EN 27027. An emulsion of the invention generally has a turbidity of from 50 NTU to 1000 NTU, depending on its specific composition. A low turbidity is generally one from 50 NTU to 400 NTU and particularly preferably from 50 NTU to 200 NTU. A low turbidity can be achieved with the emulsion of the invention even with the high proportions, indicated above, of a fat-soluble substance α₁), particularly in the embodiment of β-carotene.

The emulsion of the invention is distinguished in a further embodiment by its good applicability in the beverage industry, i.e. the positive properties described herein can be achieved in each case or in various combinations in particular in applications for example in the beverage industry.

When the emulsion is used in the beverage industry, a desired concentration is in particular from approximately 5 ppm to 50 ppm β-carotene, particularly preferably from approximately 10 ppm to 25 ppm β-carotene and especially from 15 ppm to 20 ppm β-carotene, in the beverage ready for consumption. The β-carotene concentration is from 15 to 18 ppm in a particularly preferred embodiment.

A further possibility on use of the emulsion in the beverage industry is for high electrolyte contents to be present. High electrolyte contents are achieved especially in critical systems such as concentrates or isotonic beverages having a considerable proportion of various salts.

It is further possible on use of the emulsion in the beverage industry in general for a low pH to be present. A low pH means in connection with the present invention in particular a pH of 6 or below, for example a pH of from 2 to 5 and especially a pH of from 2 to 3.

A further possibility on use of the emulsion in the beverage industry is to carry out a pasteurization. By pasteurization is meant in connection with the present invention for example a heat treatment at below 100° C., preferably 60° C. to 90° C., for a period of from 60 s to 120 s.

Besides the pasteurization, also possible in a further embodiment of the present invention are further technical steps customary in particular in the beverage industry, such as homogenization with the emulsion of the invention, without an emulsion of the invention losing its high stability or its high color intensity and its high stability and its high color intensity.

It is further possible in a particular embodiment of the emulsifier system of the invention to obtain protein-free emulsions in particular for the beverage industry. Proteins may generally comprise allergens, so that in a particular embodiment the emulsion of the invention is free of proteins. To this end, in general no proteins are used as further possible emulsifiers or stabilizers.

In a further embodiment of the present invention, the emulsion of the invention is very compatible and can be combined in particular with the formulations typically employed in the beverage industry. By compatibility is meant in connection with the present invention a small interaction with the components normally used in the beverage industry, in particular with the stabilizers used therein. The usual formulations of the beverage industry are accordingly not impaired, or are negligibly impaired, by the emulsion of the invention. The emulsion of the invention is thus capable of excellent integration into the formulations of the beverage industry.

Depending on the area of application, further components or additives can be added to the emulsion of the invention. Components α), β), γ) and the optionally possible components in this case together amount to 100% by weight based on the emulsion.

The emulsion of the invention may, in addition to the emulsifier system of the invention, also comprise one or more different, for example two or three low molecular weight, emulsifiers. A suitable additional emulsifier is, for example, lecithin in a proportion of from 5% by weight to 30% by weight based on the emulsion, but especially 10% by weight to 20% by weight based on the emulsion.

To prepare an emulsion with particular long-term stability, creaming or sedimentation of the dispersed phase must be prevented, and its tendency to coalesce must be reduced even further. The emulsifier system of the invention may in this case comprise, in addition to the emulsifiers a), b), c), also further components which are designated stabilizers in connection with the present invention. The stabilizers are preferably high molecular weight compounds having a molecular weight of 1000 or more. They are preferably employed if the emulsion of the invention is added to beverages or other aqueous solutions in order to stabilize the disperse phase in addition to the emulsifier system of the invention. The preferably high molecular weight stabilizers are thus added in particular on use of the emulsion of the invention, for example by adding the emulsion of the invention to the formulations of the beverage industry.

Typical examples of stabilizers are gum arabic, gelatin, casein, caseinate, pectin, dextrin, carob flour, guar gum, xanthan, or vegetable proteins such as soybean proteins, which may optionally be hydrolyzed, and mixtures thereof. However, it is also possible to employ polyvinyl alcohol, polyvinylpyrrolidone, methylcellulose, carboxymethylcellulose, hydroxypropylcellulose and alginates. Modified starches are also suitable. These are starch modification products prepared by technological, i.e. physical or chemical, means. Preferred stabilizers are gelatins such as ox, pig and fish gelatin, vegetable proteins, pectin, casein, caseinate and gum arabic and modified starches. Particularly preferred stabilizers are modified starches.

It is also possible to add antioxidants to the emulsion of the invention. Suitable examples are BHA (butylhydroxyanisole), BHT (butylhydroxytoluene), ascorbyl palmitate, without departing from the limits according to the invention on the amount of ascorbyl palmitate, or gallic esters. Also suitable are vitamin C and rosemary extract.

Antioxidants can be added to the emulsion of the invention in amounts of from 0.5% by weight to 10% by weight, preferably from 1% by weight to 5% by weight, where the % by weight data are based in each case on the emulsion of the invention.

The antioxidant effect of ascorbyl palmitate can, as is known, be increased further by additionally admixing tocopherol with the disperse phase α). The proportion of tocopherol is preferably from 1% by weight to 4% by weight based on the emulsion of the invention.

The emulsion of the invention is distinguished in a further embodiment of the present invention by its bacteriostatic effect. By this is meant the prevention of bacterial growth and bacterial multiplication without killing the bacteria.

Mixing of the components of the emulsion of the invention can take place immediately before addition of the emulsion to a foodstuff, to a beverage, to an animal feed, to a cosmetic or to a pharmaceutical. However, the emulsion of the invention can also be initially dispensed into containers and stored after its preparation, in order to be added as required to a foodstuff, to a beverage, to an animal feed, to a cosmetic or to a pharmaceutical.

The emulsion of the invention comprises in a preferred embodiment 5% by weight to 15% by weight of a fat-soluble substance α₁) in the embodiment of β-carotene, 0.5% by weight to 1% by weight of an emulsifier system γ) of the invention in the embodiment having 20% by weight to 30% by weight of an emulsifier b) in the embodiment of an ethoxylated sorbitan fatty acid ester of the structural formula

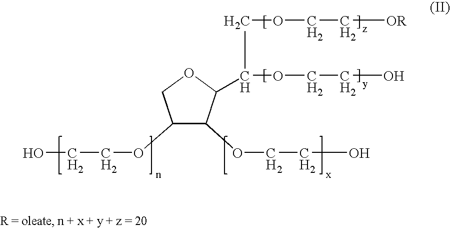

and with 20% by weight to 30% by weight of an emulsifier c) in the embodiment of a sugar fatty acid ester having the chemical structural formula

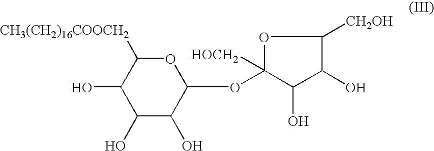

and with an amount of ascorbyl palmitate such that the total reaches 100% by weight based on the emulsifier system, and an amount of the dispersion medium β) in the embodiment of a glycerol-water ratio of from 95:5 to 85:15 such that components α₁), β) and γ) amount in total to 100%.

Processes for preparing the emulsion of the invention are known to the skilled worker. The emulsion of the invention can for example be prepared in two steps:
preemulsification by emulsifying the fat-soluble substance α₁) in the dispersion medium β) to prepare a crude emulsion and
final emulsification to prepare a final emulsion.

The emulsion of the invention is preferably prepared using a rotor/stator system and with a subsequent emulsification in a high-pressure homogenizer in one passage or in a plurality of passages. The final emulsification can take place by passing the crude emulsion one or more times, e.g. two or three times, for example through a high-pressure homogenizer. The homogenization generally takes place under a pressure of from 200 bar to 1200 bar, preferably from 600 bar to 800 bar. The speed of rotation is generally from 6000 to 10 000 revolutions per minute, but preferably from 8000 to 10 000 revolutions per minute.

The emulsion of the invention can be used for diverse purposes, especially for coloring, for vitaminization, especially provitamin A or as antioxidant in human nutrition, especially in beverages, particularly preferably in soft drinks, vitamin juices or sports drinks. Use in animal nutrition, in the cosmetic industry or in the pharmaceutical industry is also possible.

The emulsion of the invention is outstandingly suitable for easy and accurate dosage for addition for example of vitamins to liquid human or animal food products or, in the case of β-carotene, for coloring beverages, e.g. lemonades.

The present invention also relates to a human foodstuff, an animal feed product, a cosmetic or a pharmaceutical which comprises an emulsifier system of the invention or an emulsion of the invention.

### EXAMPLES

The following substances and the following processes were employed for the examples below.

| | |
|---|---|
| Fat- | β-carotene |
| soluble | |
| substance | |
| α₁₁) | |
| Emulsifier | ascorbyl palmitate |
| a₁) | |
| Emulsifierb₁) | 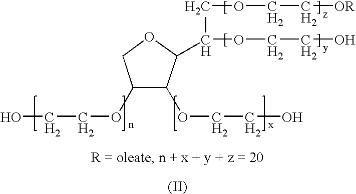 |
| Emulsifierc₁) | 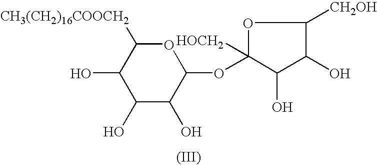 |

The relative color intensity E1/1 was measured using an HP 8452A instrument manufactured by Hewlett Packard and set at from 200 nm to 800 nm. The E1/1 value defines the specific extinction of a 1.0% strength aqueous emulsion in a 1 cm cuvette at the absorption maximum.

The UV/VIS spectra were recorded using an HP8452A manufactured by Hewlett Packard and set at from 200 nm to 800 nm.

The average particle size of the emulsion of the invention was measured via a mass-based weight distribution using a photon correlation spectrometer at a wavelength of 632.8 nm.

### Example 1

### Effect of Emulsifier Systems Compared with the Individual Emulsifiers in Emulsions with β-carotene

Disperse phase α₁) comprising:
14.43 g of β-carotene α₁₁)
28.60 g of

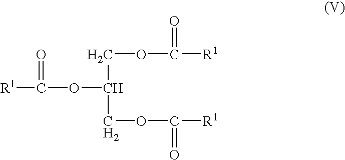

0.97 g alpha-tocopherol

Dispersion medium β₁):
34 g of deionized water
412 g of glycerol
   and
emulsifier 4 g
6 g of 1 M NaOH

The emulsions were prepared as follows. The disperse phase α₁) was heated at approximately 170° C. until the β-carotene α₁₁) had dissolved. The disperse phase α₁) was then added to the dispersion medium β₁) and homogenized using an Ultra Turrax at 10 000 revolutions per minute for approximately 15 seconds. The temperature of the emulsion was approximately 70° C. These crude emulsions were then finally emulsified in a microfluidizer manufactured by Microfluids at 800 bar in three repeats in order to obtain small droplets with a narrow size distribution of the disperse phase α₁).

TABLE 1

| | | | | Ultra Turrax | | | Microfluidizer | | |
|---|---|---|---|---|---|---|---|---|---|
| | | | | | Active | | | Active | |
| | | | | | substance | Relative color | | substance | Relative color |
| | | | | Size | content | intensity | Size | content | intensity |
| | Emulsifier | | | [nm] | [wt. %]** | [E 1/1] | [nm] | [wt. %]** | [E 1/1] |
| C1* | a₁) | | | 292 | 2.54 | 142 | 90 | 2.46 | 187 |
| C2 | | b₁) | | 318 | 2.63 | 131 | 98 | 2.43 | 190 |
| C3 | | | c₁) | | | | 469 | 1.82 | 84 |
| Emulsifier system of | a₁) | b₁) | c₁) | 315 | 2.37 | 144 | 74 | 2.42 | 190 |
| the invention | | | | | | | | | |

*Comparative test
**Based on the emulsion

2 g of emulsifier a₁), 1 g of emulsifier b₁) and 1 g of emulsifier c₁) were used for the emulsifier system of the invention. The emulsion of the invention was distinguished by a very high relative color intensity E1/1 and by a very brilliant and clear coloring. It additionally showed very good stability at room temperature of 23° C. over the observation period of four months. The results are summarized in Table 1.

### Example 2

### Concentrated Emulsions with β-carotene

Disperse phase α₂):
28.86 g of β-carotene
57.20 g of

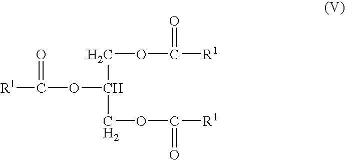

1.94 g of alpha-tocopherol

Dispersion medium β₂):
36 g of cation-exchange water
412 g of glycerol
   and
Emulsifier 4 g of emulsifier system of the invention
6 g of 1M NaOH

The emulsion was prepared by the process described in Example 1. The ratio of the contents of emulsifiers a₁), b₁) and c₁) in the emulsifier system of the invention was 2:1:1.

The commercially available concentrates showed a β-carotene concentration of approximately 2.4% by weight based on the emulsion. The emulsions prepared with the above formula had a higher β-carotene content with a constant content of the emulsifier system of the invention. The results are summarized in Table 2.

TABLE 2

| | | Ultra Turrax | | | Microfluidizer | | |
|---|---|---|---|---|---|---|---|
| | β-Carotene | | Active substance | Relative color | | Active substance | Relative color |
| | Weight | Size | content | intensity | Size | content | intensity |
| | [wt. %] | [nm] | [wt. %]** | [E 1/1] | [nm] | [wt. %]** | [E 1/1] |
| P1* | 2.9 | 270 | 2.35 | 148 | 67 | 2.4 | 189 |
| P2 | 5.3 | | | | 99 | 4.2 | 187 |
| P3 | 9.1 | 358 | 6.8 | 135 | 155 | 7.54 | 179 |

*emulsion of the invention
**based on the emulsion

The UV/VIS spectra of the emulsion P3 showed negligible loss of color intensity after 28 days.

### Example 3

### Concentrated Emulsions with β-carotene

An emulsion of the invention had the following composition, the % by weight data being based on the emulsion:

| | |
|---|---|
| β-Carotene α₁₁) | 12.1% by weight |
| 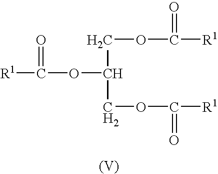 | 24.0% by weight |
| Glycerol β₄) | 57.4% by weight |
| Water β₄) | 5.04% by weight |
| Ascorbyl palmitate a₁) | 0.28% by weight |
| NaOH | 0.03% by weight |
| Tocopherol | 0.8% by weight |
| Emulsifier b₁) | 0.14% by weight |
| Emulsifier c₁) | 0.14% by weight |

The emulsion of the invention detailed above was tested in sports drinks with the stabilizers xanthan or alternatively with carboxymethylcellulose. For this purpose, the emulsion of the invention detailed above and having a content of 12% by weight of carotenoid based on the emulsion was diluted to 15 ppm to give a clear yellow sports beverage with the composition 2.0 g of stabilizer, 932 g of water, 0.57 g of phosphate salt, 0.49 g of Na citrate 5.5 H₂O, 0.8 g of NaCl, 0.15 g of Na benzoate, 0.2 g of K sorbate, 2.5 g of citric acid, 61 g of sugar and 0.15 g of ascorbic acid. A heat treatment was then carried out at 90° C. for 10 minutes. The UV/VIS absorption spectra before and after the heat treatment are virtually identical between 300 nm and 800 nm wavelength. No substantial shift in color from yellow to yellowish red was evident.

Examples 1 to 3 prove that the emulsion of the invention with the emulsifier system of the invention is very suitable for complying with the requirements of the beverage industry.

1-15. (canceled)

16. An emulsifier system comprising
a) an ascorbic ester with long-chain fatty acids,
b) an ethoxylated sorbitan fatty acid ester and
c) a sugar fatty acid ester.

17. The emulsifier system according to claim 16, comprising
a) 30 to 70% by weight ascorbic ester with long-chain fatty acids,
b) 10 to 50% by weight ethoxylated sorbitan fatty acid ester and
c) 10 to 50% by weight sugar fatty acid ester,
where the % by weight data are based in each case on the emulsifier system.

18. An emulsion comprising
α) a disperse phase comprising a fat-soluble substance α₁),
β) glycerol or glycerol mixed with water as dispersion medium and
γ) an emulsifier system according to claim 16.

19. The emulsion according to claim 18, in which the fat-soluble substance α₁) is vitamin A, D, E or K or derivatives thereof a xanthophyll, a carotenoid or a glyceride of polyunsaturated fatty acids.

20. The emulsion according to claim 18, in which the fat-soluble substance α₁) is β-carotene.

21. The emulsion according to claim 18, in which the content of the fat-soluble substance α₁) is from 2 to 40% by weight based on the emulsion.

22. The emulsion according to claim 18, in which the dispersion medium β) amounts to from 40 to 95% by weight based on the emulsion, and in which the ratio of glycerol and water is from 100:0 to 50:50.

23. The emulsion according to claim 18, in which the amount of the emulsifier system γ) is from 0.2 to 10% by weight based on the emulsion.

24. The emulsion according to claim 18, in which the average particle size of the disperse phase α) is 500 nm or less.

25. The emulsion according to claim 18, in which a further emulsifier is present.

26. The emulsion according to claim 18, in which a stabilizer is present.

27. A process for preparing the emulsion according to claim 18 by means of a preemulsification and by means of a subsequent final emulsification.

28. A process for preparing the emulsion according to claim 18 using a rotor/stator system and by means of a subsequent emulsification in a high-pressure homogenizer.

29. A human food or animal food product comprising the emulsifier system according to claim 16.

30. A human food or animal food product comprising the emulsion according to claim 18.

31. A cosmetic or pharmaceutical composition comprising the emulsifier system according to claim 16.

32. A cosmetic or pharmaceutical composition comprising the emulsion according to claim 18.
